(19) United States
(12) Patent Application Publication
(10) Pub. No.: US 2026/0174520 A1
(43) Pub. Date: Jun. 25, 2026

(54) ARRANGEMENT OF AN ELECTROSURGICAL INSTRUMENT

(71) Applicant: CMR Surgical Limited, Cambridge (GB)

(72) Inventors: Andrew Thomas Lawrence, Bedfordshire (GB); Juho Pekka Piiparinen, Cambridge (GB)

(73) Assignee: CMR Surgical Limited, Cambridge, OT (GB)

(21) Appl. No.: 19/124,642

(22) PCT Filed: Oct. 30, 2023
(86) PCT No.: PCT/GB2023/052829

§ 371 Date: Apr. 25, 2025

(30) Foreign Application Priority Data
Oct. 31, 2022 (GB) 2216133.5

(51) Int. Cl. A61B 34/00; A61B 17/00; A61B 17/068; A61B 17/072; A61B 17/29; A61B 17/32; A61B 18/14; A61B 34/30; A61B 34/37

(52) U.S. Cl. CPC A61B 34/71; A61B 17/068; A61B 17/07207; A61B 17/29; A61B 17/320092; A61B 18/1445; A61B 34/30; A61B 34/37; A61B 2017/00477; A61B 2017/2927; A61B 2017/2929

(57) ABSTRACT

A robotic electrosurgical instrument includes a shaft, an end effector including opposing first and second end effector elements, a first joint drivable by a first driving element that is constrained around the first joint, a second joint permitting the first end effector element to rotate about a second axis that is transverse to both the first axis and the longitudinal axis of the shaft, the end effector including an insulating component that covers a proximal end of the first end effector element, the insulating component including a first groove that houses the second driving element as it extends around the second joint, and an electrical cable configured to provide electrical current to an electrical component of the first end effector element.

### BACKGROUND OF THE INVENTION

This disclosure relates to a robotic electrosurgical instrument.

Electrosurgery is continually becoming more frequently used within the field of surgical robotics due to the enhanced functionality and reduced blood loss advantages achieved through the use of electrosurgical instruments. Electrosurgery is a term used to define surgical operations that are performed using instruments that are powered by a high frequency alternating electrical current that is used to heat surgical tissue.

Electrosurgical instruments that are attached to surgical robots typically comprise an end effector that is connected to a shaft via one or more articulations, or joints. The end effector may be expected to adopt a number of different rotational configurations during its use in a surgical procedure. The end effector of an instrument is typically supplied with electrical current by one or more electrical cables that are connected to the end effector of the instrument, the cables receiving current from an electrical power source that is comprised within or connected to the surgical robot.

There are various areas of development for robotic electrosurgical instruments. A significant area of development is in the configuration of electrical cables through the electrosurgical instrument so that those cables are able to withstand the different strains and configurations that are effected during robotic surgery. This is particularly relevant in view of the consistently increasing diameters of electrical cables; these diameters are widened to meet the increased power requirements of electrosurgical instruments.

### SUMMARY OF THE INVENTION

According to a first aspect, there is provided a robotic electrosurgical instrument comprising: a shaft; an end effector comprising opposing first and second end effector elements; a first joint drivable by a first driving element that is constrained around the first joint, the first joint permitting the end effector to rotate relative to the shaft about a first axis; a second joint permitting the first end effector element to rotate about a second axis that is transverse to both the first axis and the longitudinal axis of the shaft; and an electrical cable configured to provide electrical current to an electrical component of the first end effector element, the electrical cable extending by more than 45 degrees around the second joint such that it rotates about the second joint with the first end effector element.

The first end effector element may be driven about the second joint by a second driving element, the passage of the second driving element through the instrument defining a first path that extends around the second joint.

The passage of the electrical cable through the instrument may define a second path that is parallel to and offset from the first path around the second joint.

The first and second paths may be located on the same side of the longitudinal axis of the shaft.

The end effector may further comprise an insulating component that covers a proximal end of the first end effector element, the insulating component comprising a first groove that houses the second driving element as it extends around the second joint.

The insulating component may further comprise a second groove that houses the electrical cable, the second groove allowing the electrical cable to extend around the second joint.

The second groove may extend over an exterior surface of the insulating component.

The electrical cable may be connected to the electrical component at a connection point, and the connection point may be proximal to the second axis.

The second driving element may be secured to the second joint at a securing point, and the securing point may be offset from the longitudinal axis of the end effector element.

The second driving element may be secured to the second joint by a securing means and the insulating component may comprise a recess for the securing means, the recess being offset from the longitudinal axis of the end effector element.

The insulating component may comprise a first end that extends on a first side of the longitudinal axis of the end effector element and a second end that extends on a second side of the longitudinal axis of the end effector element, and the second groove may extend around the first end and the recess may be located on the second end.

The securing point may be offset from the longitudinal axis of the end effector element by between 10 and 90 degrees.

The securing point may be offset from the longitudinal axis of the end effector element by 45 degrees.

The insulating component may be composed of a single part.

The insulating component may be comprised of two separate parts: a first part that is located on a first side of the end effector element; and a second part that is located on a second side of the end effector element.

The first end effector element may be rotatable about the second joint and the second end effector element may be independently rotatable relative to the shaft about a third axis by means of a third joint.

The electrical component may be a first electrical component comprised within the first end effector element and the electrical cable may be a first electrical cable configured to provide electrical current to the first electrical component, and the surgical instrument may further comprise a second electrical cable configured to provide electrical current to a second electrical component of the second end effector element, the second electrical cable extending around the third joint such that it rotates about the third joint with the second end effector element.

The instrument may further comprise a first insulating component that covers a proximal end of the first end effector element and a second insulating component that covers a proximal end of the second end effector element.

The second joint may be drivable by a pair of driving elements comprising the second driving element and a further driving element.

The path of the electrical cable may be at least partially circumferential around the second joint.

The insulating component may be made from PEEK or polyimide.

The insulating component may be connected to the electrical component using injection moulding.

The insulating component may further comprise a cut out provides access to the connection point when the electrical cable is located within the insulating component.

The electrical connector may be accompanied by a non-conducting coupling feature that provides stress relief at the connection point.

The path of the electrical cable through the insulating component may extend parallel to the longitudinal axis of the shaft.

The electrical cable may extend more than 90 degrees around the second joint.

The insulating component may comprise one or more protrusions, the one or more protrusions being configured to, as the first and second end effector elements rotate away from the longitudinal axis of the shaft, interfere with the body of the shaft, thereby limiting the rotation of the end effector elements.

The instrument may be configured to be connected to a surgical robot.

### BRIEF DESCRIPTION ON THE FIGURES

The present invention will now be described by way of example with reference to the accompanying drawings. In the drawings:

FIG. 1 illustrates a surgical robot;

FIG. 2 illustrates a first exemplary robotic surgical instrument for use with a surgical robot;

FIG. 3 further illustrates the exemplary robotic surgical instrument of FIG. 2;

FIG. 4 further illustrates the exemplary robotic surgical instrument of FIGS. 2 and 3;

FIG. 5 illustrates an insulating component for use in a robotic surgical instrument;

FIG. 6 illustrates a second exemplary robotic surgical instrument;

FIG. 7 further illustrates the exemplary robotic surgical instrument of FIG. 6;

FIGS. 8A and 8B illustrate an end effector element for use in the robotic surgical instrument of FIGS. 6 and 7;

FIG. 9 further illustrates the exemplary robotic surgical instrument of FIGS. 6 and 7;

FIG. 10 further illustrates the exemplary robotic surgical instrument of FIGS. 6, 7 and 9;

FIG. 11 illustrates an exemplary arrangement of a robotic surgical instrument and its insulating component;

FIG. 12 further illustrates the exemplary arrangement of FIG. 11.

### DETAILED DESCRIPTION

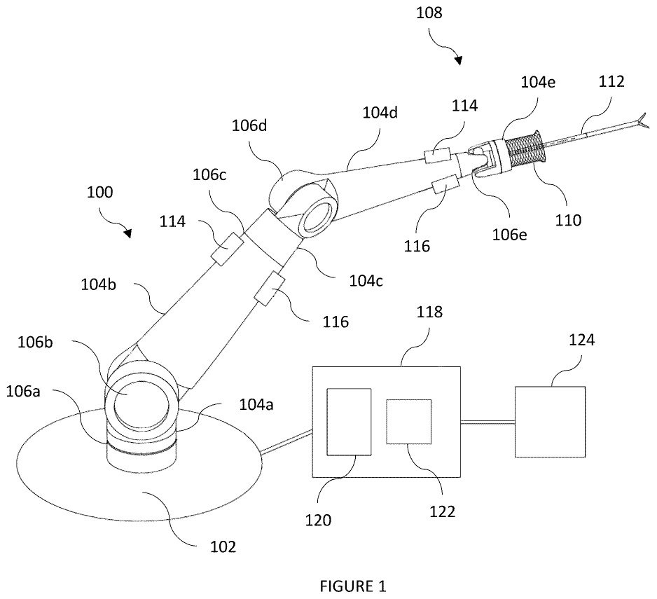

FIG. 1 illustrates a surgical robot having an arm 100 which extends from a base unit 102. The arm comprises a plurality of rigid limbs 104a-e which are coupled by a plurality of joints 106a-e. The joints 106a-e are configured to apply motion to the limbs. The limb that is closest to the base 102 is the most proximal limb 104a and is coupled to the base by a proximal joint 106a. The remaining limbs of the arm are each coupled in series by a joint of the plurality of joints 106b-e. A wrist 108 may comprise four individual revolute joints. The wrist 108 couples one limb (104d) to the most distal limb (104e) of the arm. The most distal limb 104e carries an attachment 110 for a surgical instrument 112. Each joint 106a-e of the arm 100 has one or more drive sources 114 which can be operated to cause rotational motion at the respective joint. Each drive source 114 is connected to its respective joint 106a-e by a drivetrain which transfers power from the drive source to the joint. In one example, the drive sources 114 are motors. The drive sources 114 may alternatively be hydraulic actuators, or any other suitable means. Each joint 106a-e further comprises one or more configuration and/or force (or torque) sensors 116 which provides sensory information regarding the current configuration and/or force at that joint. In addition to configuration and/or force sensory data, the one or more sensors 116 may additionally provide information regarding sensed temperature, current or pressure (such as hydraulic pressure).

The arm terminates in an attachment for interfacing with the surgical instrument 112. The surgical instrument has a diameter less than 8 mm. The surgical instrument may have a 5 mm diameter. The surgical instrument may have a diameter which is less than 5 mm. The surgical instrument comprises an end effector for performing an operation. The end effector may take any suitable form. For example, the end effector may be smooth jaws, serrated jaws, a gripper, a pair of sheers, a needle for suturing, a camera, a laser, a knife, a stapler, a cauteriser or a suctioner. The surgical instrument further comprises an instrument shaft and an articulation located between the instrument shaft and the end effector. The articulation comprises one or more joints that permit the end effector to move relative to the shaft of the instrument. The joints in the articulation are actuated by driving elements. These driving elements are secured at the other end of the instrument shaft to interface elements of an instrument interface. The driving elements are elongate elements that extend from the joints in the articulation through the shaft to the instrument interface. Each driving element can be flexed transverse to its longitudinal axis in the specified regions. In an example, the driving elements may be cables.

The diameter of the surgical instrument may be the diameter of the profile of the articulation. The diameter of the profile of the articulation may match or be narrower than the diameter of the shaft. The attachment comprises a drive assembly for driving articulation of the instrument. Movable interface elements of the drive assembly interface mechanically engage corresponding movable interface elements of the instrument interface in order to transfer drive from the robot arm to the instrument. Thus, the robot arm transfers drive to the end effector as follows: movement of a drive assembly interface element moves an instrument interface element which moves a driving element which moves a joint of the articulation which moves the end effector.

Controllers for the drive sources 114 and sensors 116 are distributed within the robot arm 100. The controllers are connected via a communication bus to a control unit 118. The control unit 118 comprises a processor 120 and a memory 122. The memory 122 stores, in a non-transient way, software that is executable by the processor 120 to control the operation of the drive sources 114 to cause the arm 100 to operate. In particular, the software can control the processor 120 to cause the drive sources (for example via distributed controllers) to drive in dependence on inputs from the sensors 116 and from a surgeon command interface 124.

The surgical instrument may be an electrosurgical instrument. The term “electrosurgical instrument” within the context of this application is used to refer to an instrument that has an end effector with an electrical component to which electrical current is to be provided for the correct operation of the surgical instrument. For example, an electrosurgical instrument can perform electrosurgery, electrocautery, diathermy or any other form of surgical procedure involving the operation of an electrical component in the end effector, e.g., for heating of surgical tissue using electrical current. The end effector(s) of an electrosurgical instrument may be similar to those of surgical instruments that are not capable of performing electrosurgery. For example, the end effector of an electrosurgical instrument may be smooth jaws, serrated jaws, a pair of shears, a knife, or a cauteriser. As it is suitable for attachment to a surgical robot, the electrosurgical instrument may be referred to as a robotic electrosurgical instrument.

If the end effector comprises one or more electrically conducting components (e.g., metal components such as metal jaws or a metal blade) then these one or more electrically conducting components may be the “electrical component(s)” to which electrical current is to be provided. For example, the end effector of an electrosurgical instrument may comprise an electrical component configured to receive electrical current and to transform that current into an alternative type of energy that is used to generate heat. The alternative type of energy may be radio frequency (RF) energy, microwave energy, ultrasound energy or energy of another suitable wavelength that is able to heat surgical tissue. The electrical component may be an electrode or a type of emitter of energy waves. The type of wave emitted by the electrical component is dependent on the frequency of the electrical current that is provided to the end effector. As another example, the end effector of an electrosurgical instrument may comprise an electrical component configured to receive electrical current and to conduct that current so that it passes into the surgical tissue of the patient, wherein when the electrical current flows through the surgical tissue it generates heat. An end effector may comprise more than one type of electrical component. For example, an end effector may comprise both an emitter of microwaves and an electrode. The different types of electrical components may be used for different purposes. For example, a first type of electrical component may be used to cut surgical tissue and second type of electrical component may be used to seal this tissue. Where the end effector comprises more than one type of electrical component, the electrical cables required to supply electrical current to each electrical component may be of the same type or of different types, depending on the requirements for the respective types of energy emitted by the components.

The instrument is supplied with electrical current by at least one electrical cable that extends along (e.g., through) the shaft. The electrical cable is attached at its first end to the instrument and at its second end to a source of electrical current (e.g., alternating current), that may be part of and located at the base of the surgical robot, for example. In an alternative example, the source of electrical current may be a standalone unit that is separate to the surgical robot. The electrosurgical instrument further comprises an electrical connector 230 that provides an electrical connection between the electrical cable and the electrical component of the end effector(s).

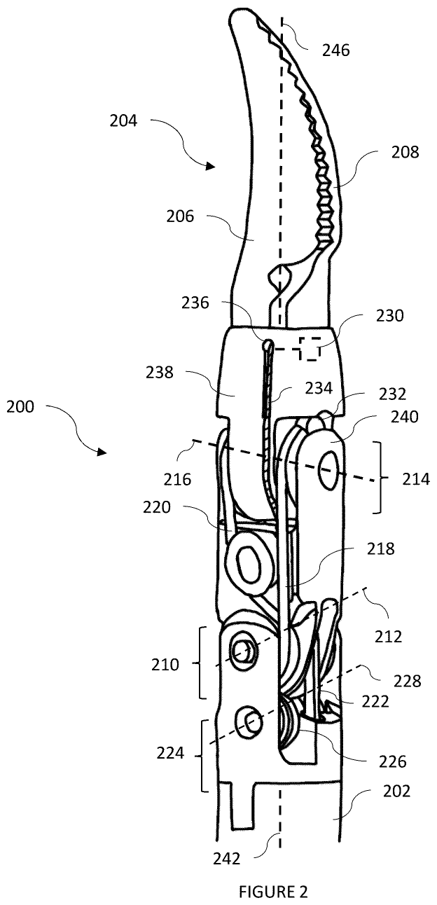

An example of the distal end of a robotic electrosurgical instrument 200 is illustrated in FIG. 2. The robotic electrosurgical instrument 200 is configured to be connected to a surgical robot. The instrument comprises a shaft 202 at its proximal end (i.e., the end closest to the connection to a robot arm) and an end effector 204 at a distal end that opposes the proximal end. The end effector 204 has a pair of end effector elements 206, 208. The end effector 204 is connected to the distal end of the shaft 202 of the instrument by an articulation. The shaft 202 is connected at its proximal end to an interface for attaching to a robot arm. The articulation comprises joints 210, 214, 224 that permit movement of the end effector 204 relative to the shaft 202.

The shaft 202 terminates at its distal end at a first joint 210. The first joint 210 is comprised within the articulation. The first joint 210 permits the end effector 204 to rotate about a first axis 212. The first joint 210 may be referred to as a rotational joint. A rotational joint shall for the purposes of this application be defined as a joint that allows two bodies to rotate relative to each other about a common axis. A rotational joint may comprise a plurality of components. The articulation comprises a supporting body 240. At a first end, the supporting body 240 is connected to the shaft 202 by the first joint 210. At a second end opposing the first end, the supporting body 240 is connected to the end effector 204 by at least a second joint 214. The instrument illustrated in FIG. 2 is in a straight configuration. In this configuration, the end effector 204 is aligned with the shaft 202. That is, in the straight configuration the longitudinal axis 246 of the end effector is coincident with longitudinal axis 242 of the shaft.

The second joint 214 permits the end effector to rotate about a second axis. More specifically, the second joint 214 permits the first end effector element 206 of the end effector 204 to rotate about a second axis 216. The second axis 216 may be transverse to the longitudinal axis 242 of the shaft 202. The second axis 216 may be perpendicular to the longitudinal axis of the shaft. A third joint (not illustrated) permits the second end effector element 208 of the end effector to rotate about a third axis (not illustrated). The third axis may also be transverse to the longitudinal axis 242 of the shaft. The third axis may also be parallel to the longitudinal axis 242 of the shaft. The further third may be parallel to the second axis 216. In the example illustrated in FIG. 2, the second and further axes are the same axis, i.e. they are collinear. However, in alternative examples, the third axis is not the same as the second axis. For example, the third axis may be parallel to but offset from the second axis 216. The offset may be in a direction defined by (e.g., along) the longitudinal axis 242 of the shaft. The offset may be in a direction that is not defined with respect to (e.g., not along) the longitudinal axis 242 of the shaft.

The first end effector element 208 and the second end effector element 206 may be independently rotatable about the second and third axes respectively because of the second and third joints. The end effector elements may be rotated in the same direction or different directions by the second and third joints. The second axis 216 is transverse to the first axis 212. The second axis 216 may be perpendicular to the first axis 212. The third axis may also be transverse and/or parallel to the first axis 212. The second joint 214 and third joint permit the end effector elements 206, 208 to rotate relative to the supporting body about the second and third axes 216.

The surgical instrument may further comprise a fourth joint 224. The fourth joint 224 may comprise at least one pulley 226. The fourth joint is located relative to the first joint 210 so as to ensure that the components that drive the first joint are retained in contact with the first joint 210. The pulleys of the fourth joint 224 may rotate about a fourth axis 228. The fourth axis 228 may be parallel to the first axis 212. The fourth axis 228 is offset from the first axis 212 along the longitudinal axis 242 of the shaft.

Each joint of the instrument is drivable by at least one driving element. Each joint of the instrument may be drivable by a pair of driving elements. Each joint of the instrument may be independently driven. The first joint 210 is drivable by at least one driving element 222. The second joint is drivable by at least one driving element 218. The third joint is drivable by at least one driving element 220. Thus, the first, second and third joints of the instrument are independently driven. The driving elements are elongate elements which extend from the joints in the articulation through the shaft to the instrument interface. Suitably, each driving element can be flexed laterally to its main extent at least in those regions where it engages the internal components of the articulation and instrument interface. In other words, each driving element can be flexed transverse to its longitudinal axis in those specific regions. This flexibility enables the driving elements to wrap around the internal structure of the instrument. The driving elements may be wholly flexible transverse to their longitudinal axes. The driving elements are not flexible along their main extents. The driving elements resist compression and tension forces applied along their length. In other words, the driving elements resist compression and tension forces acting in the direction of their longitudinal axes. Thus, the driving elements are able to transfer drive from the instrument interface to the joints. The driving elements may be cables.

The first joint 210 is drivable by a first driving element 222. The first driving element 222 extends at least partially around the first joint 210 so as to drive the first joint. That is, the first driving element 222 is constrained at least partially around the first joint. The first joint 210 may be drivable by a pair of driving elements comprising the first driving element 222 and a further driving element (not illustrated). The second joint 214 is drivable by a second driving element 218. In other words, the second driving element 218 is configured to drive the second joint 214. The elongate nature of the driving elements is such that the passage of each element through the instrument defines a path that extends at least partially around a relevant joint of the instrument. For example, the passage of the second driving element 218 through the instrument defines a first path that extends at least partially around the second joint 214. The second joint 214 may be drivable by a pair of driving elements comprising the second driving element 218 and a further driving element (not illustrated). Similarly, the third joint (not illustrated) may be drivable by a third driving element 220. The third joint may be drivable by a pair of further driving elements comprising the third driving element 220 and a further driving element (not illustrated). In some examples, the second (and/or further) driving element may be used to drive the third joint. Similarly, the third (and/or second further) driving element may be used to drive the second joint. In some examples, the second (and/or second further) driving elements and the third (and/or third further) driving elements may be used to drive the first joint. The one or more driving elements configured to drive each joint of the instrument may be secured to their corresponding joint. For example, the second driving element(s) 218 may comprise a ball feature, or crimp, 232 that is secured to the second joint 214. This ensures that when the driving elements(s) are driven, the drive is transferred to motion of the joint about the second axis 216. A corresponding ball feature is secured to each of the third joint and first joints to ensure that drive is transferred to motion of those joints about their respective axes. The driving elements may be secured to their respective joints via any alternatively suitable means.

The end effector elements 206, 208 are illustrated in FIG. 2 as being a pair of opposing serrated jaws. However, the end effector elements may take any alternatively suitable form such as smooth jaws, a pair of shears or a pair of blades of a gripping tool. Whilst the end effector of the instrument illustrated in FIG. 2 comprises two end effector elements, it is appreciated that in alternative examples the instrument may comprise a single end effector element, or more than two end effector elements.

The instrument further comprises at least one electrical cable 234. In some examples, the instrument may comprise a single electrical cable 234. The single electrical cable 234 provides electrical energy to the end effector 204. In other examples, the instrument may comprise two electrical cables. In these examples, each electrical cable may supply electrical energy to a respective electrical component of an end effector comprising two end effector elements. The electrical cable 234 is configured to provide electrical current to the end effector. The electrical cable 234 is connected to the end effector at a first end by an electrical connector 230. The electrical cable 234 may be connected at a second end to the driving elements of the surgical instrument. More specifically, the electrical cable 234 may be connected at its second end to spokes of the instrument. The spokes are rigid tubes that increase the stiffness of the driving elements. The spokes are located within the shaft 202 of the instrument. The spokes are located proximally of the end effector 204 of the instrument.

The electrical connector 230 may plug directly into the end effector 204. The electrical connector 230 may provide a fixed electrical connection between the electrical cable 234 and the end effector 204. That is, when connected to the end effector 204, the end effector 204 may not be able to move independently of the electrical connector. Alternatively, the electrical connector 230 may provide a sliding electrical connection between the electrical cable 234 and the end effector 204. That is, the end effector 204 may be able to move independently of the electrical connector when the connector is connected to the end effector 204. The connector may be any suitable means of providing an electrical connection between the electrical cable and the end effector element. The electrical connector may be accompanied by a non-conducting coupling feature that provides stress relief at the point at which the electrical connector is connected to the end effector.

The end effector comprises at least one electrical component (e.g., the metal jaws of the end effector elements 206 and 208) configured to receive electrical current from the electrical cable 234 and, for example, transform that current into energy that is used to generate heat. Energy from the electrical component is transferred to a patient via the end effector element 206, 208. In one example, the electrical component is an electrode. Where the electrical component is an electrode, the electrode is configured to heat surgical tissue by ohmic heating when electrons pass through the tissue. Alternatively, if the electrical component is an emitter of energy waves, the waves are transferred to a part of the end effector element that is configured to contact the body of a patient. The waves are in turn configured to heat and vaporise the water content of surgical tissue. The waves generated by the emitter may for example be radio frequency (RF) waves, microwaves, infrared waves, infrared waves, ultrasound waves or waves of any other suitable frequency that can be used to heat organic tissue during a surgical procedure. The type of wave emitted may be dependent on the frequency of current that is provided to the end effector. The instrument also comprises an insulating component 238 configured to insulate the electrically charged components of the instrument such as the electrical connector and the electrical component from other parts of the surgical instrument and from the patient. The insulating component 238 may comprise a groove 236 within which the electrical cable sits. The groove 236 may define a path for the electrical cable 234 from the second/third joints to the end effector elements of the end effector 204.

Where the instrument comprises a single electrical component, it is described as a monopolar instrument. For a monopolar instrument, the single electrical component of the electrosurgical instrument may be an electrode that is configured to contact the tissue of a patient and heat the tissue at a first location on the patient. The electrode of the monopolar instrument may be referred to as an “active” electrode. A second electrode may be external to the instrument and connected to a second location on the patient, and to disperse current from the active electrode from the patient. The second electrode may be referred to as a “dispersive” electrode. The monopolar instrument requires a single electrical cable to provide electrical current to its single electrical component. Thus, in turn, a monopolar instrument requires a single electrical connector to provide an electrical connection between its electrical cable and electrical component.

In an alternative example, such as the example illustrated in FIG. 2, the instrument may comprise a pair of electrical components. In this example, the electrosurgical instrument is described as a bipolar instrument. A first electrical component may be positioned on a first side of the end effector, and a second electrical component may be positioned on a second side of the end effector that opposes the first side. Where an end effector comprises two end effector elements, a first electrical component may be (or may be attached to) the first end effector element and a second electrical component may be (or may be attached to) the second end effector element. Where the end effector comprises two end effector elements, alternating energy waves may oscillate between the two end effector elements when the electrical components are charged, heating the intervening tissue by oscillation of intracellular ions.

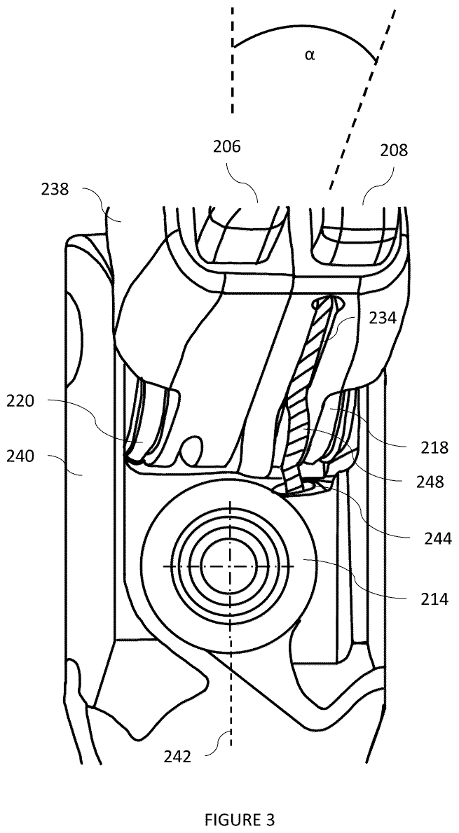

It is mentioned above that the driving elements of the surgical instrument 200 are attached to the end effectors at their distal end by a ball feature, or crimp (e.g., crimp 232), or by any alternatively suitable means. The electrical cables of the instrument are attached to the end effectors at their distal end by an electrical connector (e.g., electrical connector 230). It can be seen from FIG. 2 that the electrical connector 230 is located distally to the crimp 232 along the surgical instrument. Thus, the path length of the electrical cable 234 from the spoke of the driving element (which is the point of attachment of the electrical cable) through the instrument is different to the path length of the second driving element 218 through the instrument from the spoke. More specifically, the path length of the electrical cable from the spoke to its end effector is longer than the corresponding path length of the driving element from the spoke to the end effector. A problem associated with this difference in path lengths is illustrated in FIG. 3. In FIG. 3, a portion the instrument 200 is illustrated in a rotated pose. In this rotated pose, the longitudinal axis 246 of the end effector is positioned at an angle α with respect to the longitudinal axis 242 of the shaft. In other words, the configuration of the end effector is angled with respect to the shaft. In this angled configuration, the driving elements 218, 220 remain taught around the joints of the instrument. However, the path length of the electrical cable is longer than the corresponding path length of the driving element and so the electrical cable may experience buckling in this pose. Buckling within the context of this application is defined as the deformation of a component (i.e., the electrical cable) under load. The deformation of the electrical cable may otherwise be referred to the bending or flexion of the cable outside of its defined path. The buckling of the electrical cable in FIG. 3 is illustrated by the deformation 248, which causes path of the electrical cable to jut out from the groove 236 in the electrical insulation within which the cable is meant to be housed.

An additional problem with the configuration of the surgical instrument illustrated in FIG. 2 is that the electrical cable experiences friction as it passes through the distal end of the instrument. The distal end of the instrument in this context refers to the combination of the supporting body 240 and the end effector 204. In FIG. 3, at the distal end of the instrument, the electrical cable 234 passes through the supporting body 240 and out of an opening 244 in the supporting body towards the end effector 204. The electrical cables of the instrument 200 are formed of an inner core of electrically conductive material that is surrounded by an external casing of insulating material. When the instrument is in a rotated pose such as the one illustrated in FIG. 3 the electrical cable 234 may rub against the opening 244, leading to the wearing down of the insulating material of the cable. The friction between the electrical cable 234 and the opening 244 of the supporting body may cause the path of the cable through the instrument to deviate from its expected path, which also results in buckling. This friction also means that the cable may eventually fatigue to the point of failure. Although the cable can fatigue at any point along its length, it is most likely to fail at its connection point.

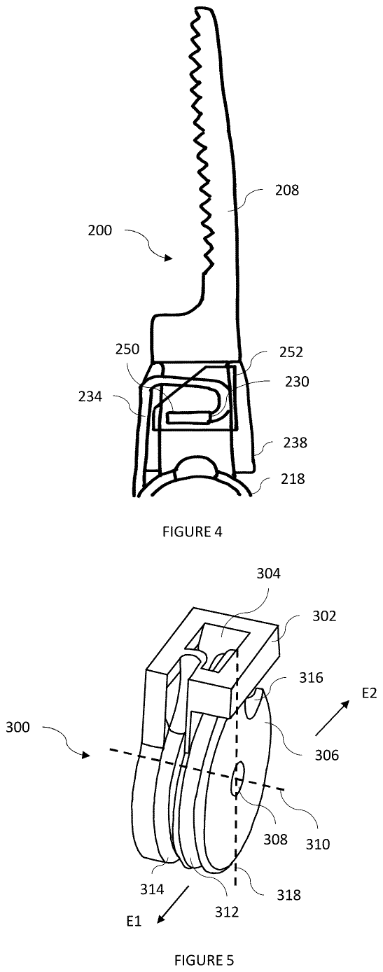

A further problem with the configuration of the surgical instrument in FIG. 2 is illustrated in FIG. 4. FIG. 4 illustrates a connection point 250 within the instrument at which the electrical connector 230 connects to an end effector 208 of the instrument. The electrical connector 230 and the distal end of the electrical cable 234 may be held in place within the insulating component 238 by a cover plate 252. The configuration of the distal end of the electrical cable 234 within the insulating component is such that it bends over on itself within the insulating component. The configuration of the insulating component 234 in FIGS. 2 to 4 is as an overmould that covers the proximal ends of the end effector elements of the instrument (i.e., the ends closest to the robot arm). Where the instrument comprises multiple end effector elements, the proximal ends of each of these end effector elements may be covered by the sole insulating component 234. The configuration of the insulating component 234 in this way is limiting as it restricts the independent rotation of the end effector elements about the second and third joints.

There is a need for an improved method of routing the electrical cable through the instrument. At the same time as preventing the cable from bending, the instrument must also be configured so that a suitable creepage distance between the connection point 250 and the joints of the instrument is maintained. The creepage distance is the shortest distance along a surface (e.g., along the longitudinal axis of the instrument) of an insulating material between two conductive paths. By ensuring a sufficient creepage distance between the connection point and the second/third joints, the transmittal of electrical energy from the electrical cable to the proximal end of the instrument (and to the patient) can be prevented.

In order to overcome the abovementioned problems, the insulating component(s) of the surgical instrument may be reconfigured as illustrated in FIG. 5. The insulating component 300 in FIG. 5 may be composed of a single part. That is, the insulating component 300 may be comprised of a single piece of material. The insulating component 300 may be manufactured using injection moulding. The insulating component 300 may be joined to an end effector element of the instrument using injection moulding. The use of injection moulding to manufacture the insulating component is described in further detail below. The insulating component 300 is configured to cover a proximal end of the end effector. More specifically, the insulating component 300 is configured to cover a proximal end of an end effector element of the end effector. The proximal end of the end effector element is the end that is closest to the shaft of the surgical instrument (and therefore the robot arm). The electrical connector of the end effector, which connects an electrical cable to an electrical component of the end effector, is located in the proximal end of the end effector. By covering the proximal end of the end effector element, the insulating component 300 covers the electrical connector. Thus, the insulating component provides a layer of electrical insulation between the electrically conducting components of the end effector and the environment located externally to these components. It also prevents the proximal end of the end effector from short circuiting, and prevents electrical energy from being conducted through the driving elements to the proximal end of the instrument (and potentially the robot arm). More specifically, the insulating component electrically insulates the electrically conducting components of the instrument from the body of a patient during a surgical procedure.

The insulating component 300 may have a geometry that matches that of the end effector element and/or the second joint of the instrument. The insulating component 300 may comprise a first portion 302 from which a first part of the end effector element extends. The first part of the end effector element comprises a tool configured to interact with the body of a patient during a surgical procedure. The first part of the end effector element may extend out of an opening 304 in the first portion 302 of the insulating component. The opening 304 may be located on a distal surface of the insulating component. The distal surface of the insulating component may be the surface that is furthest from the shaft of the surgical instrument. The first portion 302 of the insulating component may be substantially cuboidal in shape. That is, the first portion 302 of the insulating component may have a cross-sectional area that is in the shape of a quadrilateral.

The insulating component 300 may further comprise a second portion 306 that is configured to rotate about the second and/or third axis of the instrument. The second and/or third axis is illustrated by reference 310 in FIG. 5. The second portion 306 of the insulating component may be configured to house a second portion of the end effector element. The second portion of the end effector element is for enabling rotation of the end effector element about a respective joint (i.e., the second/third joint) of the surgical instrument. The second portion 306 of the insulating component may be substantially cylindrical in shape. That is, the second portion 306 may comprise a length that extends along the second/third axis 310 and a substantially circular cross-sectional area that is perpendicular to that length. The second portion 306 may comprise a channel 308 that provides a means of connecting the insulating component to the second/third joint. For example, the second/third joint may comprise a pin. In this example, the channel 308 may be configured so that the pin can pass through it. The channel 308 may be configured such that the insulating component (and the end effector element to which it is attached) can rotate about the pin. Thus, the channel 308 allows the insulating component and the end effector element to rotate about the second/third joint.

The second portion 306 of the insulating component may further comprise a first groove 312. The first groove 312 may extend at least partially around the circumference of the insulating component 300. The first groove 312 may extend fully around the circumference of the insulating component 300. In other words, the first groove 312 may extend around the entirety of the circumference of the insulating component 300. The first groove 312 may be defined as a narrow depression that extends around the circumference of the insulating component 300. The first groove 312 may have a width that extends along the second/third axis 310, and a depth that extends radially towards the centre point of the circular cross-sectional area of the insulating component 300. The depth of the first groove 312 defines a path of the first groove which has a circumference that is smaller than the circumference around the edge of the insulating component 300. The first groove 312 may extend over an exterior surface of the insulating component 300.

The purpose of the first groove 312 may be to house the second driving element as it extends around the second joint. That is, where the second driving element wraps around the insulating component 300, the driving element may be at least partially housed within the first groove 312. This means that, where the driving element contacts the insulating component 300, it contacts the first groove 312 of the insulating component. The width of the first groove 312 may therefore be wider than the width (or diameter) of the second driving element. The first groove 312 may have radii that are larger than that of the driving element. Alternatively, the depth of the first groove may be greater than the width (or diameter) of the second driving element. The first groove 312 ensures that the second driving element is not displaced when it rotates around the insulating component 300. That is, the first groove is configured so that, in operation, the second driving element is constrained within the first groove. In other words, the second driving element is pulled taught about the insulating component 300 when it rotates about the second joint. As mentioned above, in some examples, the first groove may extend entirely around the circumference of the insulating component 300. In other examples, the first groove may extend partially around the circumference of the insulating component 300. For example, the first groove may extend 180 degrees around the insulating component 300. The first groove may extend around the insulating component 300 to any suitable degree. In a specific example, the first groove may extend 115 degrees around the circumference of the insulating component 300.

The insulating component 300 may further comprise a second groove 314. The second groove may extend around the first and second portions of the insulating component. The second groove 314 may be offset from the first groove along the second/third axis 310. In other words, the first groove 312 may be located at a different distance along the axis 310 to the second groove 314. As with the first groove, the second groove 314 may have a width that extends along the axis 310, and a depth that extends radially towards the centre point of the circular cross-sectional area of the insulating component. The depth of the second groove 314 defines a path of the second groove which has a circumference that is smaller than the circumference around the edge of the insulating component 300. The second groove 314 may be said to extend around an outer circumference of the insulating component 300. The insulating component 300 may comprise different circumferences at different distances along its length. The insulating component 300 may comprise a first, maximum, circumference and two other circumferences that are smaller than the first circumference. The two smaller circumferences may be located within the first and second grooves, and may be defined by the depths of the first and second grooves. As with the first groove 312, the second groove 314 may extend over an exterior surface of the insulating component 300. Thus, the first and second grooves may each define an outer circumference of the insulating component 300.

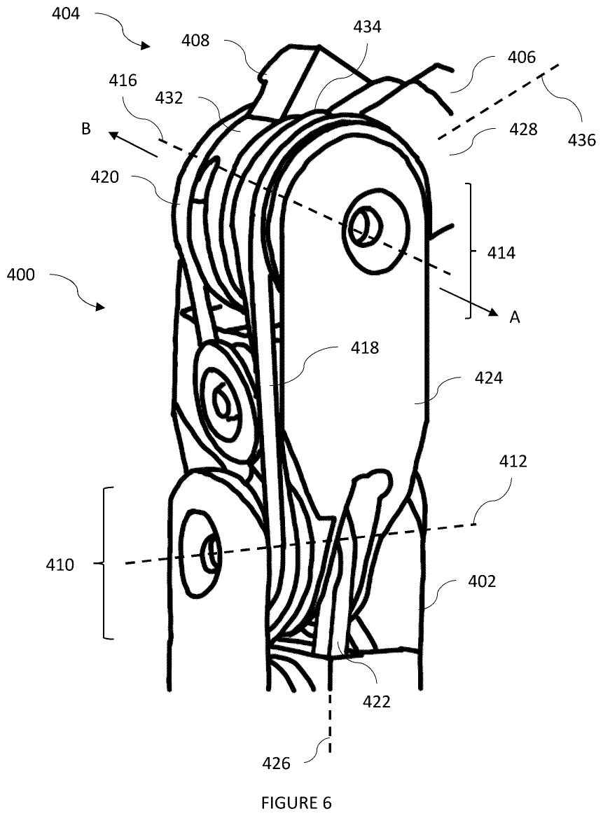

The second groove 314 may be separated from the first groove 312 along the second/third axis 310 by a non-zero distance. At the same time, the first groove 312 may be axially aligned with the second groove 314 along the axis 310. That is, the centre of the circle defining the cross-sectional area of the first groove 312 may be coincident with the centre of the circle defining the cross-sectional area of the second groove 314. In other words, the centre point of the first groove 312 may be located on the same axis as the centre point of the second groove 314. The first and second grooves may therefore extend around the same axis. More specifically, the first and second grooves may both extend around the axis 310. At the same time, the first and second grooves may be located on the same side of the longitudinal axis of the shaft when the insulating component is assembled within the instrument. This is illustrated in FIG. 6, where an instrument is illustrated comprising first and second insulating components 428, 432. In this example, the first and second grooves of the first insulating component 428 are located on the same side (in direction A) of the longitudinal axis of the shaft. As with the first groove, the second groove may extend entirely or partially around the circumference of the insulating component.

The purpose of the second groove 314 may be to house the first electrical cable 334. That is, where the first electrical cable 334 wraps around the insulating component 300, the electrical cable may be at least partially housed within the second groove 314. This means that, where the first electrical cable 334 contacts the insulating component 300, it contacts the second groove 314 of the insulating component. The second groove 314 ensures that the first electrical cable 334 is not displaced when it rotates around the second joint. That is, the second groove is configured so that, in operation, the electrical cable constrained within the second groove. In other words, the second groove 314 allows that electrical cable to extend around the second joint. The second groove 314 allows the electrical cable to extend around the second joint by a non-minimal degree. In other words, the second groove 314 allows the electrical cable to extend around the second joint by more than 45 degrees. In an example, the second groove 314 may allow the electrical cable to extend around the second joint by more than 90 degrees. The first electrical cable 334 is therefore pulled taught about the insulating component 300 when it rotates about the second joint. The path of the electrical cable may be at least partially circumferential around the second joint. That is, where the second groove extends at least partially around the circumference of the insulating component, the electrical cable extends partially circumferentially around the second joint when it is house within the second groove.

The second groove 314 may extend from the second portion 306 into the first portion 302 of the insulating component. Thus, the path of the electrical cable may be guided, by means of the second groove 314, from an outer circumference of the second portion 306 of the insulating component up to a proximal portion of the end effector element. The electrical cable may therefore be guided through the insulating component 300 from the shaft of the instrument to the end effector of the instrument via the second groove 314.

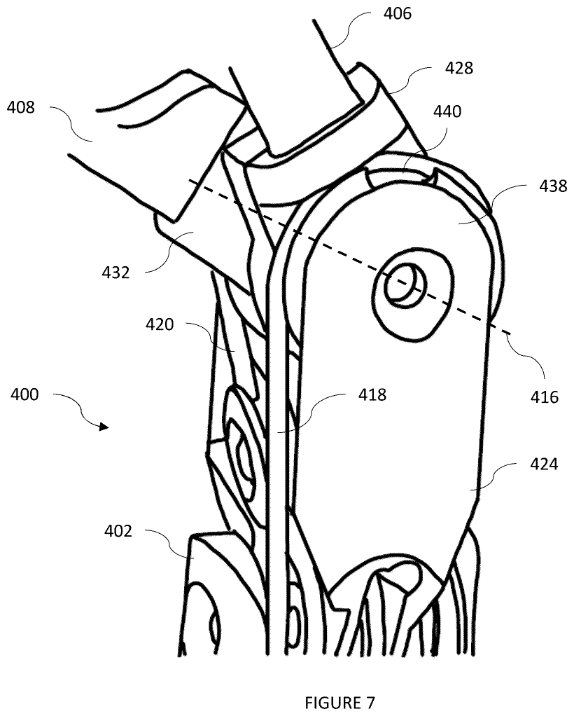

The configuration of a surgical instrument comprising at least one insulating component as illustrated in FIG. 5 is visible from FIGS. 6-9. The electrosurgical instrument 400 in FIGS. 6-9 corresponds substantially to the one illustrated in FIG. 2. The electrosurgical instrument 400 is configured to be connected to a surgical robot. The electrosurgical instrument 400 is a robotic surgical instrument that comprises a shaft 402 and an end effector 404. In FIG. 4, the end effector 404 has a pair of end effector elements 406, 408 that are the same as the end effector elements 206, 208 illustrated in FIG. 2. It will be appreciated that, in other examples, the end effector 404 may comprise a single end effector element. In FIGS. 6-9, the end effector elements 406, 408 may be first and second jaws of the end effector. The end effector 404 is connected to the distal end of the shaft 402 of the instrument by an articulation. The shaft 402 is connected at its proximal end to an interface for attaching to a robot arm. The articulation comprises joints 410, 414 that permit movement of the end effector 404 relative to the shaft 402. The articulation comprises a supporting body 424 which is the same as the supporting body 240 described with respect to FIG. 2.

The end effector 404 further comprises at least one electrical component. In one example, the at least one electrical component may be the metal jaws of the end effector elements 406 and 408. The electrical component has the same configuration and function as the electrical component described with respect to FIG. 2. In the example illustrated in FIG. 6, the instrument comprises a pair of electrical components. Thus, the instrument of FIG. 6 is a bipolar instrument. A first electrical component may be positioned on a first side of the end effector, and a second electrical component may be positioned on a second side of the end effector that opposes the first side of the end effector. Where an end effector comprises two end effector elements, a first electrical component may be (or may be attached to) the first end effector element and a second electrical component may be (or may be attached to) the second end effector element.

The instrument 400 comprises first, second, third and fourth joints that are the same as the corresponding joints described with respect to the instrument 200 in FIG. 2. Each joint of the instrument is drivable by at least one driving element. Each joint of the instrument may be drivable by a pair of driving elements. Each joint of the instrument may be independently driven. The first joint 410 is drivable by at least one driving element 422. The second joint 414 is drivable by at least one driving element 418. The third joint is drivable by at least one driving element 420. Thus, the first, second and third joints of the instrument are independently driven. The driving elements are elongate elements that extend from the joints in the articulation through the shaft to the instrument interface. Suitably, each driving element can be flexed laterally to its main extent at least in those regions where it engages the internal components of the articulation and instrument interface. In other words, each driving element can be flexed transverse to its longitudinal axis in those specific regions. This flexibility enables the driving elements to wrap around the internal structure of the instrument.

The first end effector element 408 is driven about the second joint by the second driving element 418. The elongate nature of the second driving element 418 is such that its passage through the instrument defines a first path that extends at least partially around the first joint 410. The first path of the second driving element also extends at least partially around the second joint 414. In other words, the second driving element may wrap at least partially around the first and second joints 410, 414. The passage of the second driving element through the instrument is defined by the longest dimension of the second driving element. The passage of the second driving element 418 in FIG. 6 passes through the shaft 402, out of an opening in the distal end of the shaft, around the first joint 410, and then around the second joint 414.

As with the instrument in FIG. 2, the instrument 400 is supplied with electrical current by at least one electrical cable 434 that extends along (e.g., through) the shaft 402. That is, the electrical cable is configured to provide electrical current to the electrical component of the first end effector element. The electrical cable 434 is attached at its first end to the instrument and at its second end to a source of electrical current (e.g., alternating current), that may be part of and located at the base of the surgical robot, for example. In an alternative example, the source of electrical current may be a standalone unit that is separate to the surgical robot. The electrosurgical instrument further comprises an electrical connector 430 that provides an electrical connection between the electrical cable and the electrical component of the end effector(s). The electrical connector 430 is the same as the electrical connector 230 described above with respect to FIGS. 2 and 4.

As with the driving elements, the first electrical cable 434 is of an elongate nature. The first electrical cable 434 has a length that extends in its elongate direction and a cross-sectional area that is perpendicular to its length. The cross-sectional area of the first electrical cable 434 may be of any suitable shape. In one example, the cross-sectional area is circular in shape. The cross-sectional area of the first electrical cable 434 has a width. Where the cross-sectional area of the first electrical cable 434 is circular, the width of the first electrical cable is the diameter of the first electrical cable.

The passage of the first electrical cable 434 through the instrument defines a second path. The passage of the first electrical cable 434 through the instrument is defined by the longest dimension of the electrical cable. The passage of the first electrical cable 434 through the instrument 400 passes from the spoke of the driving element, to which the cable is attached, through the body of the shaft 402, up through an opening in the distal end of the shaft (corresponding to opening 244 of the instrument 200 in FIG. 3). As illustrated in FIG. 6, the passage of the first electrical cable 434 from the distal end of the shaft 402 to the second joint 414 may extend parallel to the longitudinal axis of the supporting body 424. The passage of the electrical cable 434 from the distal end of the shaft to the second joint 414 may extend externally of the supporting body 424. The configuration of the passage of the first electrical cable from the distal end of the shaft to the second joint so that it is external to the supporting body is advantageous as it means that the electrical cable does not interfere with the path of the first driving element 422, which is located internally to the supporting body, around the first joint 410.

As with the instrument of FIG. 2, the first joint 410 of the instrument 400 permits its end effector 404 to rotate relative to the shaft about the first axis 412. The second joint 414 permits the first end effector element 406 of the instrument to rotate about the second axis 416. As described above, the second axis is transverse and/or perpendicular to both the first axis and the longitudinal axis of the shaft. The electrical cable 434 extends around the second joint 414 such that it rotates about the second joint with the first end effector element 406. In other words, the electrical cable 434 is configured to extend around the second joint as the first end effector element is articulated around the second joint. The electrical cable extends around the second joint by a non-minimal degree. That is, the electrical cable extends by more than 45 degrees around the second joint. In an example, the electrical cable may extend around the second joint by more than 90 degrees. In FIG. 6, the first end effector element 406 is in a rotated pose. That is, the longitudinal axis 436 of the first end effector element 406 is positioned at an angle with respect to the longitudinal axis 426 of the shaft. In this rotated pose, the electrical cable 434 extends smoothly around the second axis 416. That is, the electrical cable 434 does not buckle or bulge out from the second joint 414 as it extends around the second axis 416.

At the second joint 414, the passage of the electrical cable 434 follows a path that is parallel to the first path (of the second driving element 418) around the second joint. The passage of the electrical cable 434 follows a path that is parallel to the first path around at least a portion of the second joint 414. Thus, the passage of the electrical cable 434, when viewed from a plane that is perpendicular to the second axis 416, is coincident with the path of the second driving element 418 (i.e., the first path) around the second joint 414. The path of the first electrical cable 434 is also offset from the path of the second driving element 418 along the second axis 416. That is, along the second axis 416, the path of the first electrical cable 434 is separated from the path of the second driving element 418 by a non-zero distance. The first electrical cable 434 is located further along the second axis 416 than the second driving element 418. The first electrical cable 434 may be located further along the second axis 416 in first direction A. The first electrical cable 334 may alternatively be located further along the second axis 416 when viewed from a second direction B.

By configuring the passage of the electrical cable 434 so that its path around the second joint 414 is parallel to that of the second driving element 418 (e.g., using an insulating component as illustrated in FIG. 5), the difference between the path length of the electrical cable and the driving element can be reduced. By routing it around the second joint, the electrical cable can be pulled taught around that joint and excess slack in the cable can be reduced. Thus, when the instrument is articulated, there is less surplus length in the cable and so the cable does is less likely to bulge out of the instrument when the driving elements are rotated about the pulley. Thus, buckling of the electrical cable is minimised and wearing of the cable can be reduced.

The configuration of the instrument illustrated in FIGS. 6-9, which allows the path of the electrical cable 434 to extend around the second joint 414 with the second driving element 418, may allow the connection point for the electrical cable to be located proximal to the second axis 414. That is, the connection point for the electrical cable may be located close to the second axis 416. The connection point may be located adjacent to the second axis 416. The distance between the connection point and the second axis 416, along the longitudinal axis 426 of the shaft, may be less than the distance between the connection point and the tool of the end effector element.

A connection point for the instrument 400 is illustrated by the electrical connector 430 in FIG. 9. As has been described above, the connection point may be described as the point at which the electrical connector 430 connects to an end effector 406 of the instrument. The connection point may be located less than 5 mm from the second axis 416, along the longitudinal axis of the shaft. The connection point may be located less than 2.5 mm from the second axis. The connection point may be located on a first side of the longitudinal axis 436 of the end effector element.

The connection point may be located on the same side of the longitudinal axis 436 of the end effector as the side of the insulating component around which the electrical cable extends.

Thus, the electrical cable is fed in a continuous, smooth path around the second groove 314 and into the electrical component of the end effector element. In this configuration, the electrical cable does not bend back on itself. The connection of the electrical cable to the electrical component of the end effector at a connection point that is proximal to the second axis further minimises the difference in path length between the electrical cable and the driving element and therefore reduces the likelihood of buckling of the electrical cable. At the same time, the distance between the connection point for the electrical cable and the second joint allows for a sufficient creepage distance such that electrical energy is not transmitted from the cable to the proximal ends of the instrument. A further advantage is that, as the connection point is located proximal to the second axis, the length of insulating component required to cover the proximal end of the end effector element can be reduced. This means that the overall length of the end effector elements can be reduced, and therefore that more force can be exerted at the distal end of the elements for a given value of cable tension.

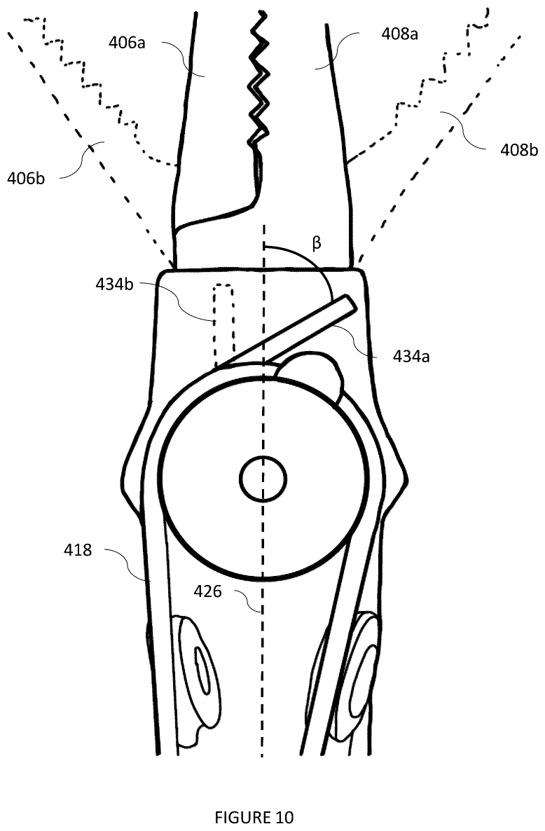

The connection point may be orientated such that the path of the electrical cable through the instrument extends in a direction that is parallel to the longitudinal axis of the shaft. The position of the electrical cable in this way is illustrated in FIG. 10. In FIG. 10, the instrument is illustrated in both a “closed” position and an “open” position in the straight configuration. In the “closed” position, assuming that the end effector is not rotated about the first axis, the longitudinal axis of the end effector element(s) is aligned with the longitudinal axis of the shaft. Where the end effector comprises two opposing end effector elements, the instrument is in a “closed” position when the end effector elements 406a, 406b are interfaced. In contrast, when the instrument is in an “open” position, the end effector elements 406b, 408b may be rotated away from each other about the second/third joints so that their interfacing surfaces are at their furthest distance from each other. In FIG. 10, for clarity, only the electrical cable 434a/b connected to the first end effector element 406a/b is illustrated.

In FIG. 10, when the instrument is in a closed position, its connection point is located within the instrument such that the electrical cable 434a is orientated at an angle β with respect to the longitudinal axis of the shaft 426. As the instrument moves between the closed position and the open position in the straight configuration, the cable may rotate such that β decreases. In the open position, β may be zero. Alternatively, the instrument may move from a straight configuration to a configuration of maximum rotation about the first axis (i.e., a configuration in which it can no longer rotate any further about the first axis). In either case, the rotation may mean that the cable 434b is moved so that it is orientated parallel to the longitudinal axis of the shaft within the insulating component. Thus, the minimum value of β, in the open position or in the maximum rotated configuration, may be zero. β may not be a negative value. The orientation of the connection point in such configurations and positions such that the path of the cable through the instrument is parallel to the longitudinal axis of the shaft in the open position is advantageous as it further minimises bending forces on the electrical cable whilst the instrument is being articulated. That is, during articulation of the end effector elements, the electrical cable does not rotate angularly past the longitudinal axis of the shaft (i.e., β does not become a negative value). This means that the cable will not bend over on itself, and thus and the likelihood of cable failure due to stress is reduced.

The instrument 400 has been described above, and illustrated in FIG. 6, as comprising at least one insulating component 300 of FIG. 5. In some examples, the insulating component 300 may enable the passage of the electrical cable to follow a path that is parallel to and offset from the path of the second driving element around the second joint. In other examples, this passage of the electrical cable around the second axis may be achieved by other means. In one such example, the second joint 414 may comprise a pulley that is rotatable about the second axis 416. In this example, the pulley may comprise at least one groove that is configured to house the electrical cable and ensure that the passage of the electrical cable around the second joint is parallel to that of the second driving element. In a further example, the supporting body may comprise a tine 438 that extends towards the distal end of the instrument, and that tine 438 may comprise at least one groove that is configured to house the electrical cable and ensure that the passage of the electrical cable around the second joint is parallel to that of the second driving element. In some examples, the passage of the electrical cable through the instrument may not be parallel to that of the passage of the second driving element. In any of the examples described above, the path of the electrical cable around the second joint may have a radius of curvature that is smaller than, or greater than, that of the second driving element. A skilled person would understand that other suitable means (e.g., other components of the surgical instrument) may be provided to guide the electrical cable such that it rotates about the second joint with the first end effector element.

In some examples, the instrument may comprise a single electrical cable 434. The single electrical cable 434 may provide electrical energy to the end effector 404. In other examples, the instrument may comprise two electrical cables. In these examples, each electrical cable may supply electrical energy to a respective electrical component of the end effector. That is, electrical cable 434 may be a first electrical cable, and the instrument may further comprise a second electrical cable configured to provide electrical current to a second electrical component of the second end effector element. Where the instrument comprises a second electrical cable, it may also comprise a second electrical connector for providing an electrical connection between the electrical cable and the electrical component of the end effector. The second electrical connector performs the same function with respect to the second electrical cable as the first electrical connector 430 performs for the first electrical cable 434. The elongate nature of the second electrical cable is the same as that of the first electrical cable 434. In the example illustrated in FIGS. 6-9, the instrument is a bipolar instrument. The instrument therefore comprises a first electrical component comprised within its first end effector element 406, and a second electrical component comprised within its second end effector element 408.

In some examples, the end effector 404 of the instrument 400 may comprise a single end effector element. In such examples, the end effector element may be a needle for suturing, a knife, a stapler or a cauteriser, or any other suitable surgical instrument. In this example, the instrument is a monopolar instrument. The instrument therefore requires a single electrical cable 434 to provide electrical current to its end effector. The passage of the electrical cable 434 through the instrument, as described above, defines a path that is offset from the first path (of the second driving element) along the second axis 416 and that extends parallel to the first path around the second joint 414. The first and second paths may be located on the same side of the longitudinal axis of the shaft when the insulating component is assembled within the instrument (e.g., in a first direction A along the second axis 416, away from the longitudinal axis of the shaft 426). The securing point of the second driving element and connection point of the electrical cable may be located on the same side of the longitudinal axis of the shaft. In other examples, the end effector may comprise opposing first and second end effector elements 406, 408. In such examples, the opposing end effector elements may be smooth jaws, serrated jaws, a gripper, a pair of sheers or any other suitable pair of elements. In these examples, the first end effector element may be rotatable about the second joint 414 and the second end effector element may be independently rotatable relative to the shaft about a third axis by means of a third joint. The third joint and the third axis may be the same as the corresponding third joint and third axis described with respect to the instrument 200 in FIG. 2.

The surgical instrument may further comprise a third driving element 420 configured to drive the third joint of the instrument. As with the second driving element 418, the length of the third driving element 420 may define a third path that extends at least partially around the third joint. The passage of the third driving element 420 through the instrument is defined by the longest dimension of the driving element. The passage of the third driving element 420 in FIG. 6 passes through the shaft 402, out of an opening in the distal end of the shaft, around the first joint 410, and then around the third joint.

Similarly to the first electrical cable, the passage of the second electrical cable through the instrument defines a fourth path. The passage of the second electrical cable through the instrument is defined by the longest dimension of the electrical cable. The passage of the second electrical cable through the instrument 400 passes from the spoke of the driving element to which it the cable attached, through the body of the shaft 402, up through an opening in the distal end of the shaft (corresponding to opening 244 of the instrument 200 in FIG. 3) and towards the first joint 410. When it reaches the first joint 410, the passage of the second electrical cable follows a path that is parallel to the third path (of the third driving element 420) around the third joint.

The passage of the second electrical cable, when viewed from a plane that is perpendicular to the second/third axis 416, may be coincident with the path of the third driving element (i.e., the third path) around the second joint 414. The path of the second electrical cable may also be offset from the path of the third driving element 420 along the third axis. That is, along the third axis, the path of the second electrical cable is separated from the path of the third driving element 420 by a non-zero distance. The second electrical cable is located further along the third axis than the third driving element 420. The second electrical cable may be located further along the second axis 416 in first direction A. The second electrical cable may alternatively be located further along the second axis 416 in a second direction B. As with the first electrical cable with respect to the second driving element, the second electrical cable may extend around the third joint such that it rotates about the third joint with the second end effector element 420.

Where the instrument comprises first and second end effector elements, it may further comprise first and second insulating components for electrically insulating the proximal parts of those end effector elements. This is illustrated in FIG. 6, where the instrument 400 comprises a first end effector element 406 and a second end effector element 408. In this example, the instrument further comprises a first insulating component 428 and a second insulating component 432. The first insulating component 428 is configured to cover a proximal end of the first end effector element 406. The second insulating component 432 is configured to cover a proximal end of the second end effector element 408. Each of the first and second insulating components may be configured as illustrated in FIG. 5. An advantage of each end effector element comprising a respective insulating component as illustrated in FIG. 6, as opposed to the configuration of the insulating component illustrated in FIG. 2, is that each insulating component can rotate with its respective end effector element about the second/third axis 416. This means that the overall range of motion of each end effector element is not limited by its insulating components, thereby improving the efficiency of the surgical instrument.

The first joint may be drivable by a pair of driving elements comprising the first driving element 322 and a first further driving element (not illustrated). It has been mentioned above that each joint in the surgical instrument may be driven, instead of by a single driving element, by a pair of driving elements. Specifically, the second joint may be drivable by a pair of driving elements comprising the second driving element 418 and a second further driving element (not illustrated). Each of the second and second further driving elements may be configured to rotate the end effector/end effector element in an opposing direction. In this way, the length of the second further driving element may define a fifth path that extends at least partially around the first and second joints. Similarly, the third joint may be drivable by a pair of driving elements comprising the third driving element 420 and a third further driving element (not illustrated). Each of the third and third further driving elements may be configured to rotate the end effector in an opposing direction. In this way, the length of the third further driving element may define a sixth path that extends at least partially around the first and third joints. Each pair of driving elements may be constructed as a single piece. Alternatively, each pair of driving elements may be constructed as separate pieces. Each pair of driving elements may be secured to its respective joint by a respecting securing means as described above.

It is mentioned above that the one or more driving elements configured to drive each joint of the instrument may be secured to their corresponding joint at a securing point. For example, the second driving element(s) may be secured to the second joint by a ball feature or crimp, or by any alternatively suitable securing means. This ensures that, when the driving elements(s) are driven, that drive is transferred to motion of the joint about the second axis 216. In the configuration of the instrument illustrated in FIG. 2, the securing point 232 for the second driving element(s) 218 is located directly above the second axis 216. This means that the centre point of the securing means, which is located at the securing point, is aligned with the second axis 216 along the longitudinal axis 242 of the shaft. Similarly, the securing point for the third driving element(s) is located directly above the third axis.

Each end effector element of the instrument may comprise a recess for housing the securing means at the securing point. Similarly, each insulating component of the instrument may comprise a recess for housing the securing means at the securing point. To form the recess in each of the end effector elements and insulating components, a volume of material must be removed from each of these components. With the insulating component illustrated in FIG. 5, the location of the first and second grooves around the external circumference of the component may be such that, if the securing point were located directly above the second axis 310, the volume of material removed to form the recess for the securing means would interfere with the path of the second groove 314 around the insulating component. This may be because the length of the securing feature, which extends parallel to the second axis, is larger than the distance between the first and second grooves along the second axis. The recess may cut through a point in the insulating component 300 at which the second groove 314 extends from the second portion 304 to the first portion 302 of the insulating component. Alternatively, the presence of the recess may result in the insulating thickness separating the securing feature from the electrically conductive components of the end effector being too small. This latter option would risk electrical energy being conducted to the driving elements of the instrument.

In order to prevent interference between the recess 316 and the second groove 314, the securing point may be located at location that is offset, in a plane aligned with the circular cross-sectional area of the insulating component, from the path of the second groove 314.

Thus, the securing point may be offset from the path of the electrical cable. More specifically, the securing point may be offset from the longitudinal axis of the end effector element 318, 436. As illustrated in FIG. 6, references to a longitudinal axis of an end effector element as described herein are to the axis that extends in a straight line from the proximal end (which is closest to the joint of rotation) to the distal end (which is furthest from the joint of rotation) of the element. This axis is unaffected by the overall shape of the end effector element. The offset means that the recess 316 for the securing means, on the insulating component 300, may be offset from longitudinal axis 318, 436 of the end effector.

The insulating component 500 may be described as comprising a first end and a second end. The first end E1 of the insulating component 500 may extend on a first side of the longitudinal axis 318, 436 of the end effector element. The second end E2 of the insulating component may extend on a second side of the longitudinal axis 318, 436 of the end effector element. As can be seen from FIG. 5, the second groove 314 may extend around the first end E1 of the insulating component. The recess 316 for the securing means (i.e., the securing point) may be located on the second side E2 of the insulating component. In other words, the securing point may be offset so that is located on an opposing end of the insulating component to the second groove 314. This means that the securing point for the driving element is located clear of the second groove 314, and its recess does not interfere with the groove or the electrical cable that is housed within that groove.

In some examples, the securing point may be offset from the longitudinal axis 318, 436 of the end effector element by between 10 and 90 degrees. In a more specific example, the securing point 316 may be offset from the longitudinal axis of the end effector element by 45 degrees. An advantage of this degree of offset is that the securing point 316 may be located far enough away from the second groove 314 that it does not interfere with the groove, or the electrical cable that is housed within that groove. The exact angle of offset between the securing point and the longitudinal axis of the end effector element that is tolerable for an instrument may be dependent on (a) the range of travel required for the end effectors of the instrument, and (b) the angle of departure of the driving elements around the second joint. The angle of departure of a driving element is the angle, relative to the longitudinal axis of the end effector, at which it departs from the circumference surrounding the axis around which it rotates. As an example, if an end effector element needs a range of motion of 120 degrees about its respective axis, and the driving element departs from the second joint at 90 degrees, then the furthest that the securing point could be offset from the longitudinal axis without compromising its range of travel would be 30 degrees. The angle of offset between the securing point and the longitudinal axis of the end effector element may be similar to the angle between the electrical cable inside the insulating component and the longitudinal axis of the end effector element. That is, these two angles may vary by less than ten degrees from each other. In one example, the two angles may be the same.

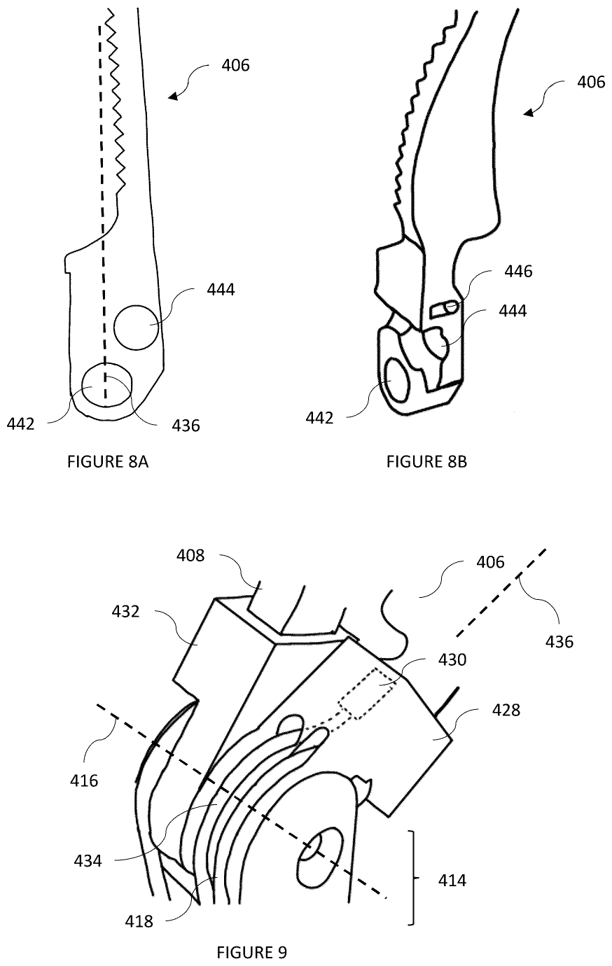

An illustration of an end effector element that may be comprised within the instrument 400 is illustrated in FIGS. 8A and 8B. The end effector element is labelled as the first end effector element 406 of the instrument 400. It may be appreciated that the second end effector element 408 of the instrument 400 may be configured correspondingly. The end effector element comprises, at its proximal end, a channel 442 for connecting the element to a joint of the instrument. The function of the channel 442 is the same as the corresponding channel 308 of the insulating component 300 illustrated in FIG. 5. The end effector element further comprises a recess 444. The recess 444 is located, in a plane that extends perpendicularly to the axis of the channel 442, at the securing point of the instrument. As with the insulating component 300, the recess 444 in the end effector element 406 is offset from the longitudinal axis 436 of the end effector element. The end effector element 406 further comprises a connection point 446. The connection point 446 comprises a groove within which an electrical cable and its electrical connector may be housed. It can be seen in FIG. 8B that the connection point 446 is proximal to the second axis, as the second axis extends through the channel 442.

The electrical connector may be any suitable means that provides an electrical connection between the electrical cable and the electrical component of the end effector(s). In one example, the electrical connector is a weld. The electrical connector may be accompanied by a non-conducting coupling feature that provides stress relief at the connection point. The non-conducting coupling does not conduct electrical energy, and thereby prevents the transmittal of electrical energy away from the electrical cable and electrical connector. In a first example, the coupling feature is merely a bend in the electrical cable that secures the electrical cable to the connection point of the instrument. The bend may be enabled via suitable manufacturing of the insulating component. That is, the insulating component may be manufactured with a further groove that provides a path for the electrical cable to bend close to the connecting point. The further groove may be significantly smaller than the first and second grooves. In a second example, the coupling feature may be a crimp that is applied to the outside of the electrical cable at the securing point. In a third example, the coupling feature may be a potting compound. A potting compound is a general-purpose type of sealant that can be used to secure the electrical cable to an internal feature of the insulating component. Use of a potting compound is advantageous as it enables the easy manufacture of the instrument. In a fourth example, the coupling feature may be a dowel that holds the electrical cable in position. The coupling feature may be any alternative component that is able to provide stress relief at the connection point.

The insulating component may further comprise a window, or cut-out, in its insulating component. The cut-out provides access to the connection point when the electrical cable is located within the insulating component. The cut-out may thereby enable the fixation of the coupling feature whilst the electrical cable is being assembled within the insulating component. For example, the potting substance and/or welding may be applied through the cut-out of the insulating component. The presence of a cut-out in the insulating component allows these manufacturing operations to be performed after the insulating component has been over moulded onto the end effector element.

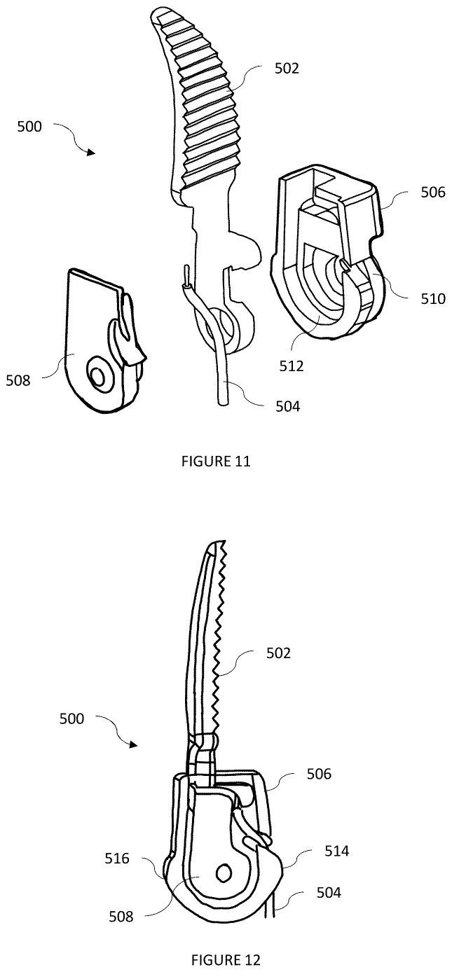

An alternative configuration of an insulating component, and its attachment to an end effector element, is illustrated in FIGS. 11 and 12. The end effector element 502 may be the same as an end effector element 406, 408 illustrated in FIG. 6. The arrangement in FIGS. 11 and 12 further comprises an electrical cable 504 which may correspond to the electrical cable 434 as illustrated in FIGS. 6-9. The insulating component in FIGS. 11 and 12 is comprised of two separate parts, a first part 506 and a second part 508. An inner shell 512 may be formed from the attachment of the first part 506 to the second part 508. The inner shell 512 is configured to hold a proximal end of the end effector element 502, as well as the electrical cable 504 for that end effector element. One of the two separate parts 506, 508 of the insulating component may comprise the inner shell 512. For example, in FIG. 11, the first part 506 comprises the inner shell. In an alternative example, the second part 508 may comprise the inner shell. In a further example, each of the first and second parts may comprise a portion of the inner shell. As with the insulating component of FIG. 5, the insulating component of FIGS. 11 and 12 comprises a groove 510 for housing an electrical cable. The insulating component may further comprise a groove for housing a driving element. Thus, the function of the insulating component in FIG. 11 may be the same as that of the corresponding component in FIG. 5. The insulating component may comprise a recess, as described above with respect to FIG. 5, for a securing point for a driving element of the instrument.

Of the two separate parts of the insulating component, a first part 506 of the component is located on a first side of the end effector element 502, and a second part 508 of the insulating component is located on a second side of the end effector element. The two separate parts of the insulating component may encapsulate the end effector element and the electrical cable between them when they are held together. The groove 510 for housing the electrical cable may be located on one of the two separate parts of the insulating component. In the example illustrated in FIG. 11, the groove 510 is located on the first part 506 of the insulating component. In an alternative example, the groove 510 may be located on the second part 508 of the insulating component. In a further example, a first portion of the groove 510 may be located on the first part 506 of the insulating component, and a second portion of the groove may be formed on the second part 508 of the insulating component. The complete groove may be formed from the attachment of the first part 506 to the second part 508.

In one example, the two separate parts of the insulating component 506, 508 may be joined together around the end effector element by gluing. In alternative examples, the two separate parts of the insulating component may be joined together using ultrasonic welding, or by using any other suitable joining means. The manufacturing of the insulating component in this way may be advantageous when compared to the manufacturing of the corresponding component illustrated in FIG. 5. More specifically, the manufacturing of the insulating component in two parts may be faster and less expensive than the manufacturing of this component as a single part. When the two separate parts of the insulating component are joined together, the electrical cable is held within the groove 510. As it is formed of two separate parts, the insulating component may be described as having a clamshell design. The insulating component of FIG. 11 may differ from that of FIG. 5 only in that it is comprised of two separate parts.

As with the configuration illustrated in FIG. 5, the configuration of the insulating component of FIGS. 11 and 12 may allow the connection point for at least a first electrical cable to be located proximal to the second axis 416 of the instrument. That is, the connection point for the electrical cable may be located close to the second axis 416. Thus, as with the insulating component 300, the insulating component 500 may allow the electrical cable to be fed in a continuous, smooth path around the second axis of the instrument and into the electrical component of the end effector element. In this configuration, the electrical cable does not bend back on itself. The connection of the electrical cable to the electrical component of the end effector at a connection point that is proximal to the second axis further minimises the difference in path length between the electrical cable and the driving element and therefore reduces the likelihood of buckling of the electrical cable.

The insulating components described herein may be manufactured from any suitable material. In some examples, the insulating components may be made of polyetheretherketone (PEEK) or polyimide. PEEK and polyimide are pharmaceutical grade materials that are long wearing and offer high strength-to-weight ratios. Where the insulating component is manufactured from PEEK or polyimide, the component may be fixed to the end effector element using injection moulding. In injection moulding, a mould for the insulating component is placed around the proximal end of an end effector element, and a heated polymer material is injected into the mould. Once it has been injected into the mould, the polymer cools and hardens, forming the shape of the insulating component around the end effector element. An advantage of the use of injection moulding to form the insulating component is that it is a straightforward and cost-efficient manufacturing process, as its moulds can be reused and material waste is limited. Injection moulding also ensures part reliability and consistency in high volume production. In another examples, the insulating component may be made of a ceramic or composite material. Such materials offer similar strength-to-weight ratios to those of the polymers described above, but can generally withstand higher temperatures. Where the insulating component is manufactured from ceramics or composites, the component may be fixed to the end effector element using gluing, heat bonding, mechanical interlocking, welding or any other suitable means.

The insulating component of FIGS. 11 and 12 further comprises protrusions 514, 516. Protrusions 514, 516 are named as such because they protrude from the otherwise uniform geometry of the insulating component. In one example, the insulating component may have a generally cuboidal geometry. In another example, the insulating component may have a generally cylindrical geometry. In both examples, the length of the insulating component extends along the second/third axis. The protrusions 514, 516 protrude outwards from the generally square/circular cross-sectional area of the insulating components. The protrusions may otherwise be referred to as knurls. The protrusions may have bump-like profiles that extend out of the width of the insulating component. The width of the insulating component is perpendicular to both the longitudinal axis of the shaft and the second/third axis. The insulating component may have a single protrusion. Alternatively, the insulating component may have two protrusions. Where the insulating component has two protrusions, one protrusion may be located on either side of the width of the insulating component. The purpose of the protrusions may be to stop the end effector elements from rotating further than they are designed to. That is, as the end effector elements rotate away from the longitudinal axis of the shaft, the protrusions may interfere with the body of the shaft, thereby limiting rotation of the elements. The limitation of motion of the end effector elements means that the amount of rotation of the electrical cables about their respective axes is limited, and therefore that the stress on the cables is minimised. In particular, the stress on the cables is minimised in the situation where the end effector elements are manually operated and damaged by a user. Although they are annotated in the component of FIGS. 11 and 12, the protrusions 514, 516 may alternatively be implemented into the insulating component of FIG. 5.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A robotic electrosurgical instrument comprising:
a shaft;
an end effector comprising opposing first and second end effector elements;
a first joint drivable by a first driving element that is constrained around the first joint, the first joint permitting the end effector to rotate relative to the shaft about a first axis;
a second joint permitting the first end effector element to rotate about a second axis that is transverse to both the first axis and the longitudinal axis of the shaft, wherein the first end effector element is driven about the second joint by a second driving element, the passage of the second driving element through the instrument defining a first path that extends around the second joint;
the end effector further comprising an insulating component that covers a proximal end of the first end effector element, the insulating component comprising a first groove that houses the second driving element as it extends around the second joint and a recess configured to house a securing means-feature to secure the second driving element to the second joint at a securing point, wherein the securing point is offset from the longitudinal axis of the end effector element; and
an electrical cable configured to provide electrical current to an electrical component of the first end effector element, the electrical cable extending by more than 45 degrees around the second joint such that it rotates about the second joint with the first end effector element.

2. The instrument of claim 1, wherein the passage of the electrical cable through the instrument defines a second path that is parallel to and offset from the first path around the second joint.

3. The instrument of claim 2, wherein the first and second paths are located on the same side of the longitudinal axis of the shaft.

4. The instrument of claim 1, wherein the insulating component further comprises a second groove that houses the electrical cable, the second groove allowing the electrical cable to extend around the second joint.

5. The instrument of claim 4, wherein the second groove extends over an exterior surface of the insulating component.

6. The instrument of claim 1, wherein the electrical cable is connected to the electrical component at a connection point, and the connection point is proximal to the second axis.

7. The instrument of claim 1, wherein the insulating component comprises a first end that extends on a first side of the longitudinal axis of the end effector element and a second end that extends on a second side of the longitudinal axis of the end effector element, and wherein the second groove extends around the first end and the recess is located on the second end.

8. The instrument of claim 1, wherein the securing point is offset from the longitudinal axis of the end effector element by between 10 and 90 degrees.

9-10. (canceled)

11. The instrument of claim 1, wherein the insulating component is comprised of two separate parts:
a first part that is located on a first side of the end effector element; and
a second part that is located on a second side of the end effector element.

12. The instrument of claim 1, wherein the first end effector element is rotatable about the second joint and the second end effector element is independently rotatable relative to the shaft about a third axis by means of a third joint.

13. The instrument of claim 12, wherein the electrical component is a first electrical component comprised within the first end effector element and the electrical cable is a first electrical cable configured to provide electrical current to the first electrical component, and wherein the surgical instrument further comprises a second electrical cable configured to provide electrical current to a second electrical component of the second end effector element, the second electrical cable extending around the third joint such that it rotates about the third joint with the second end effector element.

14. The instrument of claim 12, further comprising a first insulating component that covers a proximal end of the first end effector element and a second insulating component that covers a proximal end of the second end effector element.

15. (canceled)

16. The instrument of claim 1, wherein path of the electrical cable is at least partially circumferential around the second joint.

17. The instrument of claim 1, wherein the insulating component is made from PEEK or polyimide.

18. (canceled)

19. The instrument of claim 6, wherein the insulating component further comprises a cut out that provides access to the connection point when the electrical cable is located within the insulating component.

20. The instrument of claim 6, wherein the electrical connector is accompanied by a non-conducting coupling feature that provides stress relief at the connection point.

21. The instrument of claim 1, wherein the path of the electrical cable through the insulating component extends parallel to the longitudinal axis of the shaft.

22. The instrument of claim 1, wherein the electrical cable extends more than 90 degrees around the second joint.

23. The instrument of claim 1, wherein the insulating component comprises one or more protrusions, the one or more protrusions being configured to, as the first and second end effector elements rotate away from the longitudinal axis of the shaft, interfere with the body of the shaft, thereby limiting the rotation of the end effector elements.

24. The instrument of claim 1, wherein the instrument is configured to be connected to a surgical robot.
